(19) United States
(12) Patent Application Publication
(10) Pub. No.: US 2015/0265200 A1
(43) Pub. Date: Sep. 24, 2015

(54) Safety belt arrangements and methods for determining information with respect to the cardiac and/or respiratory activity of a user of a safety belt

(71) Applicant: TAKATA AG, Aschaffenburg (DE)

(72) Inventors: Koshan MAHDI, Berlin (DE); David Maximilian MAAS, Berlin (DE)

(21) Appl. No.: 14/340,336

(22) Filed: Jul. 24, 2014

(30) Foreign Application Priority Data
Mar. 19, 2014 (DE) 10 2014 004 202.2
Jun. 16, 2014 (DE) 10 2014 211 501.9

(51) Int. Cl. A61B 5/18; A61B 5/00; A61B 5/0205; A61B 7/02; B60W 40/08; B60R 22/48

(52) U.S. Cl. CPC A61B 5/18; B60W 40/08; B60R 22/48; A61B 5/0205; A61B 7/02; A61B 5/6893; B60W 2040/0872; A61B 5/024

(57) ABSTRACT

The invention relates to safety belt arrangements with a safety belt, a belt buckle and a belt tongue; at least one sensor region arranged at the safety belt, at the belt buckle and/or at the belt tongue, which includes at least one first sensor and at least one second sensor. At least one evaluation unit is provided for determining information with respect to the cardiac and/or respiratory activity of the user of the safety belt by using the values determined by the first and second sensors of the sensor region.

### CROSS-REFERENCE TO RELATED APPLICATION

This application claims priority to German Patent Application No. 10 2014 004 202.2 filed on Mar. 19, 2014, and to German Patent Application No. 10 2014 211 501.9 filed on Jun. 16, 2014, the entirety of which is incorporated by reference herein.

### BACKGROUND

The invention relates to safety belt arrangements for a motor vehicle and to methods for determining information with respect to the cardiac and/or respiratory activity of a user of a safety belt of a motor vehicle.

From the prior art, there are known driver assistance systems which intervene in the operating behavior of a vehicle in a corrective and optimizing manner and/or act on a driver of the vehicle. To enable a driver assistance system to intervene to the correct extent and also at the desired time, a fairly good knowledge of the current mental and/or physical state of the driver (for example his current stress level) and e.g. also other vehicle occupants is desirable. For detecting the mental and/or physical state of the driver, there are known different sensor systems for determining biometric data (vital data) of the driver of a motor vehicle, wherein information on the mental and/or physical state of the driver should be obtained from the biometric data. For example, WO 2004/110829 A1 discloses a sensor array integrated into a safety belt of a vehicle.

### SUMMARY

It is a problem underlying the invention to improve the signal quality of a sensor array which can be integrated into a vehicle.

According to an exemplary embodiment of the invention, there is provided a safety belt arrangement for a motor vehicle, comprising
a safety belt, a belt buckle, and a belt tongue;
at least one sensor region arranged at the safety belt, at the belt buckle and/or at the belt tongue, which includes:
   at least one first sensor for determining values of a quantity dependent on a cardiac and/or respiratory activity of a user of the safety belt; and
   at least one second sensor for determining values of at least one ambient quantity, wherein
the safety belt arrangement comprises at least one evaluation unit for determining information with respect to the cardiac and/or respiratory activity of the user of the safety belt by using the values determined by the first and second sensors of the sensor region.

For example, the safety belt arrangement comprises a plurality of sensor regions arranged at the safety belt, at the belt buckle and/or at the belt tongue, wherein the sensor regions each include at least one first sensor for determining values of a quantity dependent on a cardiac and/or respiratory activity of a user of the safety belt and at least one second sensor for determining values of at least one ambient quantity, and wherein the evaluation unit is formed for determining information with respect to the cardiac and/or respiratory activity of the user of the safety belt by using the values determined by the first and second sensors of at least one of the sensor regions.

It is conceivable that (e.g. exclusively) several sensor regions are provided at the safety belt. It is, however, also conceivable that at least one sensor region is provided at the safety belt and a further sensor region is provided at the belt buckle or the belt tongue.

The values determined by the second sensor primarily serve to reduce or eliminate environmental influences on a signal which is generated by the first sensor on detection of the cardiac and/or respiratory activity of the user of the safety belt. In principle, the ambient quantity which is detected by the second sensor can be any measurable quantity which has an influence on the detection of the quantity dependent on the cardiac and/or respiratory activity of the user; e.g. a temperature in the surroundings of the first sensor. The quantity determined by the second sensor in particular depends on the type of the first sensor.

It is conceivable that the signals of several second sensors of various sensor regions of the safety belt (e.g. all sensor regions) are used to process the signal of at least one first sensor. It is also possible that the information with respect to the cardiac and/or respiratory activity of the user is determined by using signals of several first sensors of various sensor regions. There can also be selected one of the first sensors or a plurality of first sensors (e.g. with reference to the signal quality of the first sensors), which is used for determining the cardiac and/or respiratory activity. For example, the signal quality depends on the position of the sensor relative to the position or body height of the user. It is possible, for example, that with a small user only one (lower) first sensor supplies a sufficiently good signal, whereas with a larger user the signal of several first sensors can be used.

For example, the first sensor of at least some of the sensor regions each is formed by a first acoustic transducer (which converts sound waves into an electrical signal, e.g. a microphone) and the second sensor each likewise is formed by an acoustic transducer (e.g. likewise a microphone), wherein the first and the second acoustic transducer are oriented along different directions, i.e. the directional characteristics of the acoustic transducers are oriented at an angle to each other.

In particular, in use of the safety belt arrangement the first acoustic transducer is directed towards the user of the safety belt arrangement, whereas the second acoustic transducer is oriented such that it chiefly detects sounds of the surroundings of the sensor region. For example, the second acoustic transducer faces the vehicle interior. Thus, breathing sounds and/or the heartbeat of the user are detected by means of the first acoustic transducer, whereas the surrounding sounds recorded by the second acoustic transducer serve the correction of the signal of the first acoustic transducer. Correspondingly, the evaluation unit in particular is formed to correct signals which are generated by the first acoustic transducer on detection of sounds which depend on the cardiac and/or respiratory activity of the user, by using surrounding sounds detected with the second acoustic transducer. Disturbances in the signal of the first sensor, which result from disturbing noises from the surroundings, thus can be removed or at least be reduced.

For example, the correction is effected by using a model of an acoustic transmission path between the first and the second acoustic transducer, wherein the model in particular takes account of the differences of the surroundings of the first and the second sensor.

According to another variant of the invention, the first sensor is an acceleration sensor or a pressure sensor. The second sensor can be a temperature sensor which detects a temperature in the surroundings of the first sensor. In particular, the evaluation unit here is formed to correct signals which are generated by the first sensor in dependence on the cardiac and/or respiratory activity of the user, by using the temperature detected with the temperature sensor. For example, thermally induced fluctuations in the signal of the first sensor can be corrected by means of the temperature sensor.

When the first sensor is a pressure sensor, the same e.g. is arranged at the safety belt such that a contact pressure which the safety belt exerts on the user can be determined. The contact pressure depends on the respiratory and/or cardiac activity of the user, so that it is possible to draw conclusions as to the respiratory and/or cardiac activity by detecting the contact pressure. In addition, by measuring the contact pressure it might be determined whether the safety belt is fastened correctly. It is also conceivable that a pressure sensor or an acceleration sensor is provided as second sensor or in addition to the first and second sensors, in order to detect disturbances, in particular disturbances which result from a movement of the user, and to take account of the same when evaluating the sensor signals.

As pressure sensor, e.g. a piezo sensor or a resistive element can be used for force determination (FSR—force sensing resistor).

In a second aspect, the invention relates to a safety belt arrangement for a motor vehicle, comprising
a safety belt, a belt buckle, and a belt tongue;
at least one sensor region arranged at the safety belt, at the belt buckle and/or at the belt tongue, which includes:
   at least one first sensor for determining values of a first quantity dependent on a cardiac and/or respiratory activity of a user of the safety belt; and
   at least one second sensor for determining values of a second quantity dependent on a cardiac and/or respiratory activity of the user of the safety belt and different from the first quantity, wherein
the safety belt arrangement comprises at least one evaluation unit for determining information with respect to the cardiac and/or respiratory activity of the user of the safety belt by using the values determined by the first and second sensors of the sensor region.

For example, the safety belt arrangement according to the second aspect of the invention comprises a plurality of sensor regions arranged at the safety belt, at the belt buckle and/or at the belt tongue, wherein the sensor regions each include at least one first sensor for determining values of a first quantity dependent on a cardiac and/or respiratory activity of a user of the safety belt and at least one second sensor for determining values of a second quantity dependent on a cardiac and/or respiratory activity of the user of the safety belt and different from the first quantity, and wherein the evaluation unit is formed for determining information with respect to the cardiac and/or respiratory activity of the user of the safety belt by using the values determined by the first and second sensors of at least one of the sensor regions.

In addition, the safety belt arrangement includes an evaluation unit for determining information with respect to the cardiac and/or respiratory activity of the user of the safety belt by using the values determined by the first and second sensors of at least one of the sensor regions.

Due to the fact that signals of different sensors (in particular of different sensor types) are evaluated, it is possible in particular to increase the reliability of the determination of the cardiac and/or respiratory activity of the user. For example, the plausibility of a signal of at least one of the first sensors or of a quantity derived from the sensor signal is checked with reference to a signal of at least one of the second sensors.

It is conceivable that the first or the second sensor is a capacitive sensor. However, the invention is not limited to a particular sensor type. Thus, the first or the second sensor also might be formed by an acoustic transducer (in particular in the form of a microphone) or by an acceleration sensor. It is also possible that the first or the second sensor is an active sensor, e.g. an ultrasonic sensor or a high-frequency sensor. According to a configuration of the second aspect of the invention, the first sensor is a capacitive sensor or an acoustic transducer and the second sensor is an acceleration sensor or an active sensor.

In particular, the evaluation unit is formed to correct a signal of the first sensor in dependence on a signal of the second sensor or to subject information concerning the cardiac and/or respiratory activity of the user, which is derived from the signal of the first sensor, to the above-mentioned plausibility check with reference to the signal of the second sensor.

In accordance with another development of the invention according to the first or the second aspect, the sensor regions each comprise a signal processing unit with which a filtration and/or amplification of a signal generated by the first and/or the second sensor is effected. It is also conceivable that a single signal processing unit is provided, which is associated to all sensor regions. Analogously, a single evaluation unit can be associated to all or at least several sensor regions. It is, however, also possible that several evaluation units are present, which each are associated to one sensor region and e.g. also are arranged adjacent to the sensors of the respective sensor region.

It is also possible that the first and/or the second sensor of the sensor regions each are arranged on a printed circuit board extending along the safety belt. For example, the first and/or the second sensor of several sensor regions are arranged on a common printed circuit board.

The evaluation unit of the sensor array according to the invention (according to the first or second aspect of the invention) in addition can be formed to determine information with respect to the body height of a user of the safety belt in dependence on the values determined by the first and/or second sensor in the sensor regions. As already mentioned above, it is conceivable for example that with a smaller user only some sensors respond, so that the body height can be inferred from the comparison of the sensor signals of sensors of different sensor regions (which are arranged one behind the other in particular along the safety belt).

Furthermore, the evaluation unit can be formed to generate a signal in dependence on the values determined by the first and/or second sensor in the sensor regions, which signal signalizes whether the safety belt is fastened correctly. When the second sensors of at least some of the sensor regions, as already mentioned above, are temperature sensors, those sensors which have no contact to the user, i.e. which are located in a region of the safety belt which is not fastened correctly, will indicate a lower temperature than sensors which are positioned close to the body of the user, i.e. are arranged in a correctly fastened region of the safety belt. Thus, e.g. the number and/or the position of those sensors which signalize a plausible temperature can provide a statement as to the fit of the safety belt.

Furthermore, the evaluation unit can be provided to generate signals which after an accident and in the presence of vehicle-based emergency call systems can provide information as to the state of health of the vehicle occupants (“post-crash notification”).

The number of the sensor regions and their distance from each other in particular is dimensioned such that substantially independent of the body height of the vehicle occupant at least one of the sensor regions or at least two sensor regions is/are located in the region of the upper body of the vehicle occupant. For example, at least five sensor regions are present.

According to another variant of the invention, the first and/or the second sensor of several of the sensor regions are formed by a microphone, wherein the sensor regions form a microphone array for generating a speech signal usable by a hands-free system. The microphone signals in particular are evaluated such that there is obtained a virtual microphone with specifiable directional characteristic.

In addition, the evaluation unit can be formed to carry out a voice analysis. For example, it can be detected whether the driver or another vehicle occupant is speaking. Furthermore, a voice analysis might provide information with respect to the mental state of the vehicle occupant, e.g. with respect to a state of excitement or other psycho-physiological features of the vehicle occupant. This information can be combined with the determined data with respect to the cardiac and/or respiratory activity of the vehicle occupant (for example for calibration or for a plausibility check).

In particular, the microphones in addition can be formed to generate a processed signal for determining the cardiac and/or respiratory activity of the user, as explained above with respect to the first aspect of the invention.

The invention also relates to a method (in particular by using a device according to the first aspect of the invention) for determining information with respect to the cardiac and/or respiratory activity of a user of a safety belt of a motor vehicle, with the following steps:
providing a safety belt, a belt buckle, a belt tongue and at least one sensor region arranged at the safety belt, the belt buckle and/or the belt tongue, including:
   at least one first sensor for determining values of a first quantity dependent on a cardiac and/or respiratory activity of a user of the safety belt; and
   at least one second sensor for determining values of at least one ambient quantity, and
determining information with respect to the cardiac and/or respiratory activity of the user of the safety belt by using the values determined by the first and second sensors of the sensor region.

In a further aspect, the invention also relates to a method (in particular by using a device according to the second aspect of the invention) for determining information with respect to the cardiac and/or respiratory activity of a user of a safety belt of a motor vehicle, with the following steps:
providing a safety belt, a belt buckle, a belt tongue and at least one sensor region arranged at the safety belt, the belt buckle and/or the belt tongue, including:
   at least one first sensor for determining values of a first quantity dependent on a cardiac and/or respiratory activity of a user of the safety belt; and
   at least one second sensor for determining values of a second quantity dependent on a cardiac and/or respiratory activity of the user of the safety belt and different from the first quantity, and
determining information with respect to the cardiac and/or respiratory activity of the user of the safety belt by using the values determined by the first and second sensors of the sensor region.

### BRIEF DESCRIPTION OF THE DRAWINGS

The invention will subsequently be explained in detail by means of exemplary embodiments with reference to the Figures:

FIG. 1A shows a safety belt arrangement according to an exemplary embodiment of the invention.

FIG. 1B shows the safety belt arrangement of FIG. 1A in use by a smaller vehicle occupant.

FIG. 2 shows the safety belt arrangement of FIGS. 1A and 1B in a perspective view.

FIG. 3A shows sensor regions of a safety belt arrangement according to the invention in a top view.

FIG. 3B shows the sensor regions of FIG. 3A in a side view.

FIG. 4 shows a safety belt of a safety belt arrangement according to the invention.

FIG. 5 shows sensor regions of another safety belt arrangement according to the invention in a top view.

FIG. 6 shows a modification of a safety belt arrangement according to the invention.

FIG. 7 shows a safety belt arrangement according to the invention with correctly fastened safety belt.

FIG. 8 shows the safety belt arrangement of FIG. 8 with not correctly fastened safety belt.

FIG. 9 shows a flow diagram for detecting a not correctly fastened safety belt.

FIG. 10 shows a flow diagram for determining information with respect to the cardiac and/or respiratory activity of a vehicle occupant according to the invention.

FIG. 11 shows a safety belt arrangement according to another exemplary embodiment of the invention.

### DETAILED DESCRIPTION

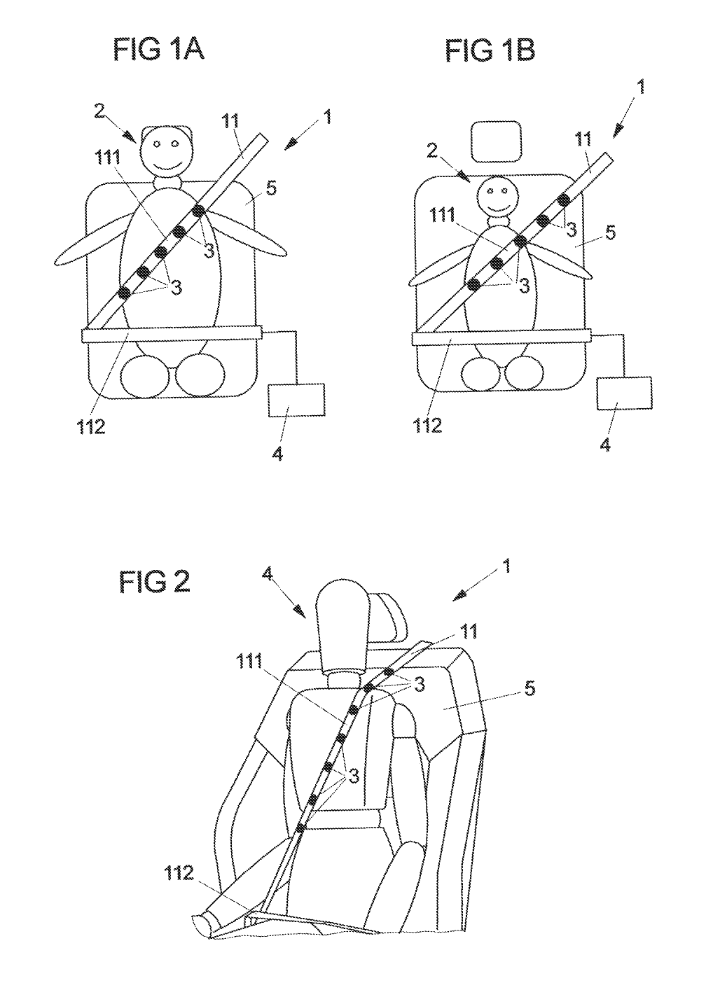

FIG. 1A shows a safety belt arrangement 1 installed in a motor vehicle with a safety belt 11 which has been fastened by a user (vehicle occupant 2). The safety belt 11 includes several sensor regions in the form of sensor units 3, which serve to obtain data with respect to the cardiac and/or respiratory activity of the vehicle occupant 2. From these data, in particular information on the mental and/or physical state of the vehicle occupant 2 should be derived and possibly a reaction of a driver assistance system should be initiated (e.g. initiation of a braking operation) and/or a warning signal should be generated (e.g. when the measured cardiac and/or respiratory activity indicates an imminent falling asleep of the vehicle occupant).

It is also conceivable that the information on the state of the vehicle occupant, which is based on the data of the sensor units 3, is combined with data of other sensor systems, e.g. with data of a camera-based system for observing the vehicle occupant and/or a system for measuring the electrodermal activity. Alternatively or in addition, it is also possible that the safety belt arrangement 1 according to the invention is used for detecting a medical case of emergency, wherein e.g. on detection of a case of emergency an alarm message automatically can be sent to an emergency station (for example via an emergency call module such as “ecall” of the vehicle).

The sensor units 3 are regularly arranged along a portion 111 of the safety belt 11, which in the fastened state of the safety belt 11 is in contact with the upper body of the vehicle occupant 2. Sensors (not shown in FIGS. 1A and 1B) of the sensor units 3 are connected with a common evaluation unit 4 which from the sensor signals determines the above-mentioned data with respect to the cardiac and/or respiratory activity of the vehicle occupant. The evaluation unit 4 can be connected with the sensors via an electric line integrated into the safety belt 11. For example, the evaluation unit 4 is accommodated in a lower region of a vehicle seat 5 and via a lap portion 112 of the safety belt 11 connected with the sensors of the sensor units 3 by means of the line integrated into the belt 11. However, a wireless connection of the sensors to the evaluation unit 4 also is conceivable.

The number of the sensor units 3 and their distance from each other is dimensioned such that substantially independent of the body height of the vehicle occupant 2 at least one of the sensor units 3 or at least two sensor units 3 is/are located in the region of (e.g. in contact with) the upper body of the vehicle occupant 2. In the exemplary embodiment of FIG. 1A, which shows a larger vehicle occupant 2, all sensor units 3 (here five sensor units) are located in the region of the upper body of the vehicle occupant 2.

FIG. 1B shows the same safety belt arrangement 1 as FIG. 1B, wherein a smaller vehicle occupant 2 (for example a child), however, is located on the vehicle seat 5. Correspondingly, not all sensor units 3 are fastened in the region of the upper body of the vehicle occupant 2, but only two sensor units arranged in the lower region of the upper body portion 111 of the safety belt 11. The presence of two sensor units 3 in the region of the upper body of the vehicle occupant 2, however, is sufficient to obtain evaluable sensor signals and reliable information with respect to the cardiac and/or respiratory activity of the vehicle occupant.

FIG. 2 shows the safety belt arrangement 1 of FIGS. 1A, 1B in a perspective representation, wherein a vehicle occupant 2 of similar size as in FIG. 1A is located on the vehicle seat 5.

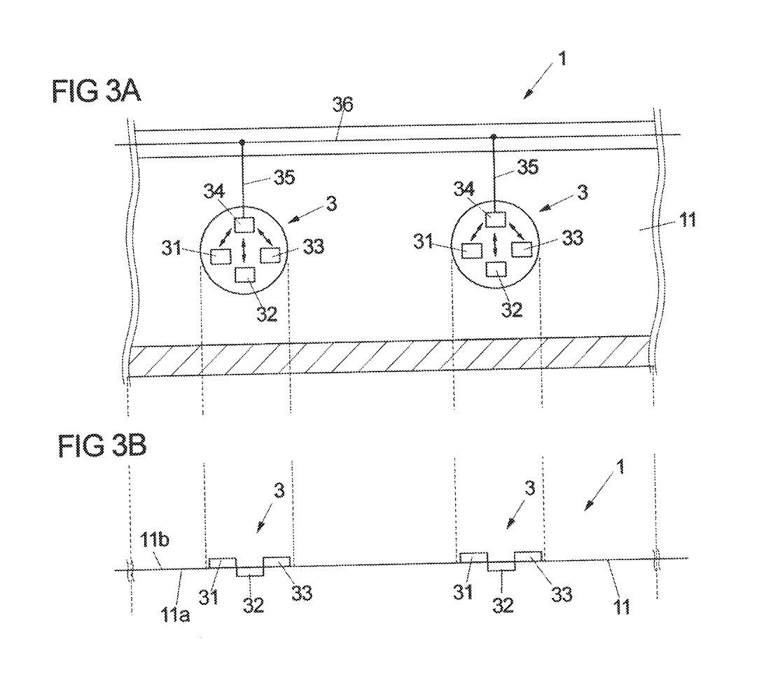

FIGS. 3A and 3B show a possible design of the sensor units 3. Accordingly, at least the two illustrated sensor units 2 each include a first, second and third sensor 31, 32, 33, which each are connected with a common signal processing unit in the form of an amplification and/or filter unit 34. The amplification and/or filter unit 34 processes the signals of the sensors 31, 32, 33 separately or jointly and forwards them to the external evaluation unit 4.

One of the sensors (the sensor 32) is arranged on an inside 11a of the safety belt 11, which after fastening the safety belt 11 faces the vehicle occupant 2. The other sensors 31, 33 are arranged on an outside 11b of the safety belt 11, i.e. on a side of the safety belt 11 which will face away from the vehicle occupant 2. It is of course also conceivable that several sensors are arranged on the inside 11a of the safety belt 11, or also that at least some sensors are at least partly located in the safety belt 11. The position of the sensors 31-33 at the safety belt 11 in particular depends on the type of the sensors. Capacitive sensors for example will be arranged on the inside 11a of the safety belt 11, since they should get in contact with the vehicle occupant 2. When microphones or active sensors are used as sensors, the same can have a certain distance to the vehicle occupant and therefore can very well be arranged on the inside 11b of the belt.

Via the amplification and/or filter unit 34 and at least one connecting line 35, the sensors 31-33 are connected with an evaluation unit (cf. FIGS. 1A and 1B) for evaluating the signals of the sensors 31-33 by means of at least one electric line 36 (e.g. in the form of a communication bus) extending along at least a section of a lateral edge of the safety belt 11. Via the electric line 36 or via a further line which likewise extends along the lateral edge of the safety belt 11, the sensors 31-33 and/or the amplification and/or filter unit 34 also can be supplied with energy. The lines 35, 36 are integrated (e.g. woven) into the belt e.g. during the manufacture of the safety belt 11. It is, however, also conceivable that the lines 35, 36 are mounted at the belt 11 subsequently, e.g. in the form of an electrically conductive coating.

At least some of the sensors 31-33 and/or the amplification and/or filter unit 34 of one of the sensor units each in particular are arranged on a common carrier (for example a printed circuit board). In particular those sensors are arranged on a common carrier, which are to be positioned on the same side of the safety belt 11. Of course, the sensor units 3 also can include more or less than the three sensors shown in FIGS. 3A and 3B. It is also conceivable that at least some of the sensor units 3 of the safety belt arrangement according to the invention are formed differently and differ e.g. in the number and/or the type of the sensors.

The evaluation unit 4 in particular is a programmed module, which e.g. carries out a signal processing for example by combination and/or comparison of the signals of the different sensors 31-33. For example, corresponding algorithms are implemented in the evaluation unit 4, for instance according to the principle of blind source separation, beam forming and/or difference formation.

The sensors 31-33 in particular are selected such that two different types of sensor are present among the same, i.e. sensors which detect values of different quantities, which depend on the cardiac and/or respiratory activity of the vehicle occupant. It is also conceivable that at least one type of sensor is present at least in duplicate, wherein the signals of the two identical sensors are combined, for example, in order to improve the signal quality.

For example, a capacitive ECG sensor (such as the sensor 32 to be arranged facing the vehicle occupant) is present among the sensors 31-33, which by capacitive coupling registers states and their changes, as well as a sensor (such as one of the sensors 31, 33) formed by an acoustic transducer (audio sensor, in particular in the form of a microphone) which detects a sound pressure from the direction of the vehicle occupant. The ECG sensor detects the electrical activity of the heart of the vehicle occupant, whereas the acoustic transducer can hear the heartbeat and the respiration of the vehicle occupant, so to speak. The ECG sensor includes e.g. an electrode of an electrically conductive material, whereby a relatively large electrode surface and at the same time a flexible electrode can be realized. The signal of the audio sensor can be ampli-fied with an audio amplifier, which provides for using smaller audio sensors.

It is also possible that a movement sensor is used, with which a movement in particular of the upper body of the vehicle occupant is detected. For example, this is an at least uniaxial acceleration sensor, wherein the main axis of the sensor in particular is orthogonal to the safety belt 11. The acceleration sensor detects a movement (vibration) of the upper body (in particular of the chest) of the vehicle occupant caused by the respiration.

As already mentioned above, the sensor units 3 each are constructed e.g. as integrated unit, i.e. the sensor components are located on a common printed circuit board or are part of a common electronic module (“system-on-chip”), which provides for a rather small form factor of the sensor units (in particular with respect to the height of the sensor units) and hence for a rather incon-spicuous and hardly disturbing arrangement of the sensor units at/in the safety belt 11. Merely the ECG electrode and the connecting lines 35, 36 would be realized as separate components.

As sensor of the sensor units 3, there might also be used an active sensor (or several active sensors), e.g. an ultrasonic or radar sensor which transmits the ultrasonic waves or radar waves to the vehicle occupant and receives reflected waves.

The combination of two different sensor types in the sensor units 3 in particular provides for a plausibility check of the signals generated by the sensors or of information derived from these signals with respect to the mental and/or physical state of the vehicle occupant. Thus, an evaluation of the sensor signals for example is possible under realistic conditions, i.e. in particular also in the case of a movement of the vehicle occupant on the vehicle seat (in particular during changes of the seating position) and/or in the case of vibrations of the vehicle.

It is also possible that at least some of the sensor units 3 include at least one sensor (ambient sensor) which detects values of at least one ambient quantity. For example, the sensor 33 is an ambient sensor. The values detected by this sensor in particular serve to correct signals of another sensor (such as the sensors 31, 32), which detects a quantity dependent on the cardiac and/or respiratory activity of the vehicle occupant. For example, the ambient sensor is a temperature sensor for detecting a temperature in the region of the respective sensor unit or a microphone for detecting disturbing sounds, as already described above.

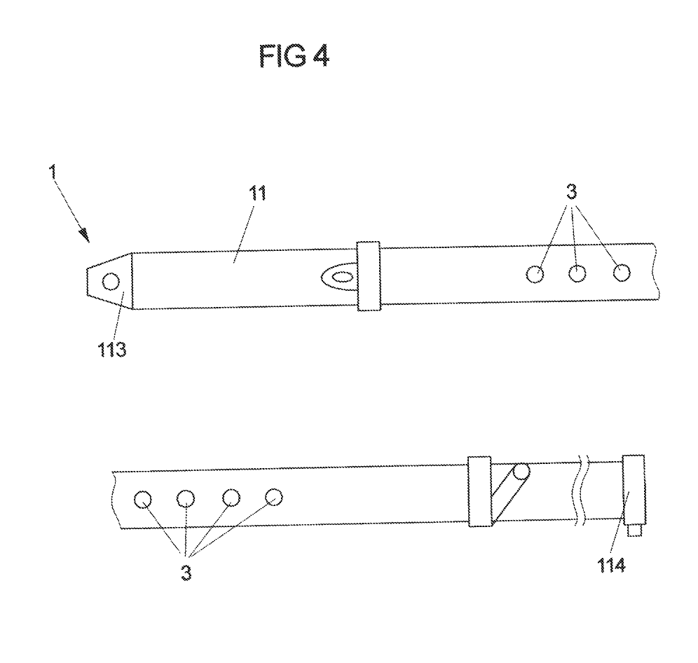

As shown in FIG. 4, the sensor units 3 in particular are positioned with a distinct distance on the one hand to a belt anchor 113 of the safety belt 11 and on the other to a retractor 114 at the upper body portion 111 of the safety belt 11. The sensor units 3 thus are not retracted as well after use of the safety belt 11. For example, the distance to the belt anchor 113 is at least 800 mm and to the retractor 114 at least 1400 mm.

From the number and the position of the sensor units 3, whose sensors provide meaningful (usable) signals after fastening the safety belt 11, the body height and hence the weight of the vehicle occupant also can be inferred. This information in turn can be used for controlling vehicle components (such as adaptive air bags).

Instead of the connection of the sensors 31-33 of the sensor units 3 via the connecting lines 35, 36 as indicated in FIG. 3A, there can also be realized a wireless connection of the sensors 31-33 with the evaluation unit 4 and/or an energy source (in particular by induction). The sensors 31-33 here each include a radio unit or are connected with a radio unit which transmits the radio signals F to a receiving unit. The receiving unit e.g. comprises an antenna 30 integrated into the safety belt 11 and extending along at least a portion of an edge of the safety belt 11. Via the antenna 30, both sensor data and energy for supplying the sensors 31-33 can be transmitted. The wireless transmission of the sensor signals and the wireless energy supply of the sensors provides e.g. for an arrangement of the sensors directly on the safety belt, without having to equip the same with electric structures. The sensors therefore can easily be integrated into the safety belt, since no line structures are required for connecting the sensors with each other and/or with the evaluation unit.

It is also conceivable that the energy for the sensors is produced locally, e.g. by utilizing heat, movement, light and/or electromagnetic waves present in the region of the sensor units 3 as energy source. Of course, a battery (e.g. of the vehicle) can also be used as energy source.

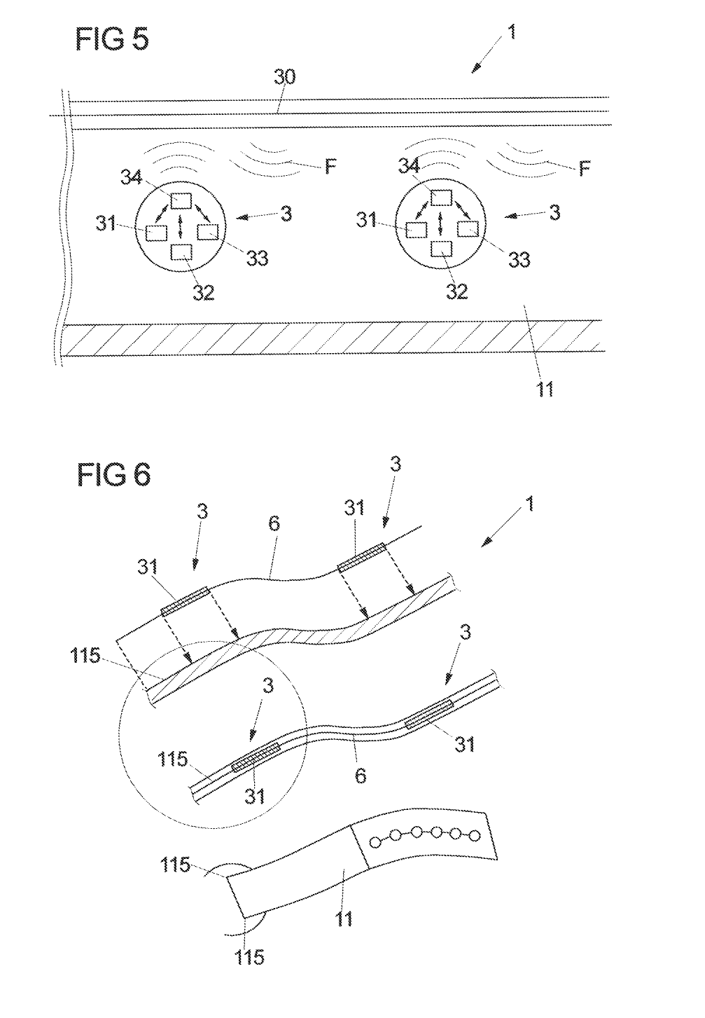

Another variant of the wire-bound transmission of the sensor data and the energy supply, which differs from FIG. 3A, is shown in FIG. 6. Here, sensors 31 of the sensor units 3 each are arranged on a flexible printed circuit board 6 (e.g. soldered to the printed circuit board 6). It is conceivable that the sensors 31 of different sensor units 3 each are arranged on separate printed circuit boards which are connected with each other via lines.

It is, however, also possible that as shown in FIG. 6 the sensors 31 of at least some of the sensor units 3 are arranged on a common printed circuit board 6. The printed circuit board 6 can be elongated (for instance like a strip), wherein the printed circuit board 6 with the sensors 31 is arranged in a tubular receptacle 115 of the safety belt 11. The tubular receptacle 115 forms a lateral edge of the safety belt 11. In the present case, the safety belt 11 includes at least two tubular receptacles 115, which form opposite lateral edges of the safety belt 11 and in each of which a continuous printed circuit board 6 with sensors 31 is arranged. It is of course also conceivable that only one tubular receptacle 115 and correspondingly only one printed circuit board 6 is present. Furthermore, each of the sensor units can of course also comprise more than the one sensor 31 shown in FIG. 6.

The printed circuit boards 6 arranged in the tubular receptacles 115 are connected with the evaluation unit of the safety belt arrangement 1. For example, the connection is made at an end fitting (e.g. the belt tongue) of the safety belt 11, wherein the printed circuit boards 6 extend e.g. up to the end fitting or a line extends in the tubular receptacles 115, via which the printed circuit boards 6 are connected with the evaluation unit.

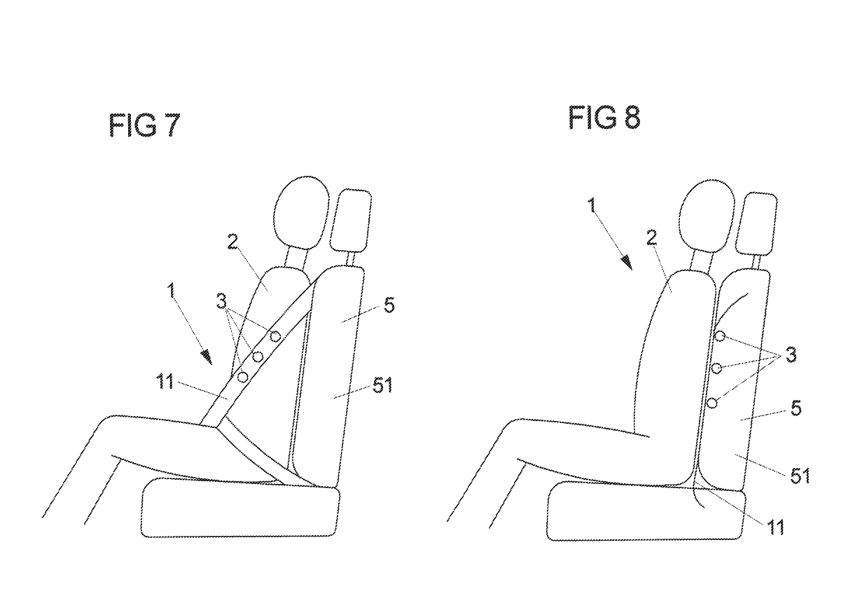

FIGS. 7 and 8 illustrate that the sensors of the safety belt arrangement 1 can also be used for monitoring the fit of the safety belt 11. This is possible in particular because the sensors, to be able to measure a quantity dependent on the cardiac and/or respiratory activity of the vehicle occupant, have a directional characteristic which—based on the correctly fastened state of the safety belt 11 (FIG. 7)—points in direction of the vehicle occupant 2 or the vehicle seat 5.

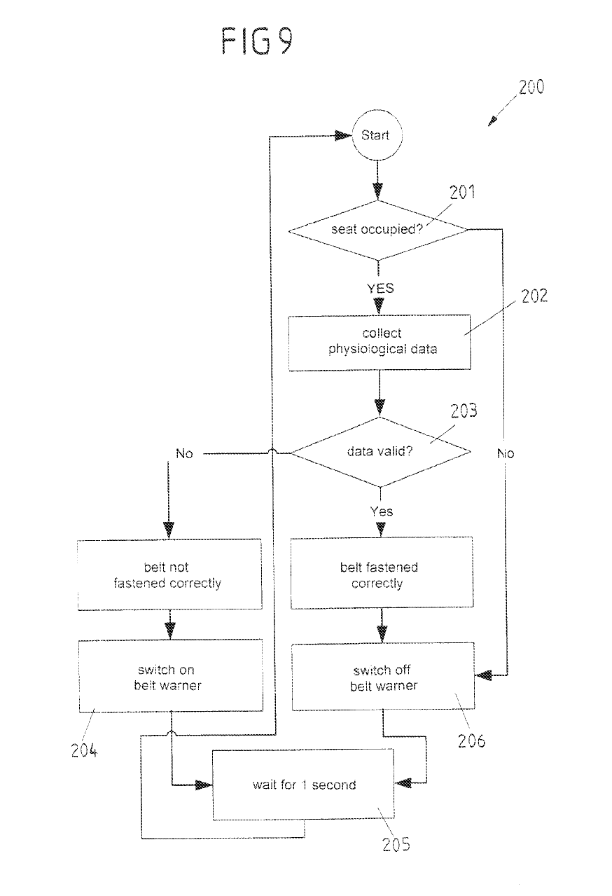

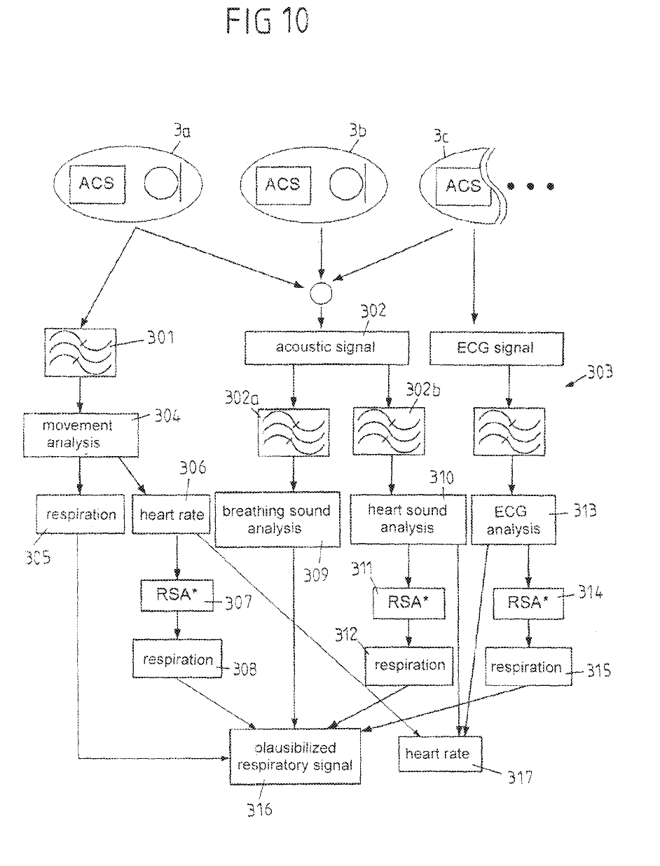

In so far, the sensor signal can be used to distinguish whether a signal (e.g. heart or breathing sound) coming from a backrest 51 of the vehicle seat 5 couples to the sensor units 3 (i.e. towards the inside of the safety belt 11) or running towards the backrest 51 couples to the sensors (i.e. towards the outside of the safety belt 11), as is shown in FIG. 8. By means of an algorithm (cf. FIG. 9) an incorrect use of the safety belt 11 correspondingly can be detected, e.g. when the same extends behind the back of the vehicle occupant 2. Thus, circumventing conventional belt usage controls, which merely check whether the belt buckle is locked correctly, can be prevented. It is also conceivable that with reference to the sensor signals it is detected whether a per-son or an object is secured with the safety belt 11.

According to FIG. 9 the belt usage control method 200 initially comprises an examination as to whether or not the vehicle seat is occupied (step 201). If this is the case, at least one quantity dependent on the cardiac and/or respiratory activity of a user of the safety belt is detected by means of the sensor units of the safety belt arrangement according to the invention (step 202). By evaluation and/or comparison of the signals of different sensors a plausibility check 203 of the sensor signals is made. When the sensor signals (sensor data) are found to be not plausible (not valid), a belt warner is activated (step 204). After a specifiable waiting time 205 (e.g. 1 second), the method is passed through again starting with step 201.

However, should the plausibility check 203 reveal that the sensor data are valid, it is concluded that the sensors are oriented correctly towards the vehicle occupant and the safety belt accordingly is fastened correctly. Thereupon, a belt warner possibly is deactivated (step 206) and the method is repeated after the waiting time 205.

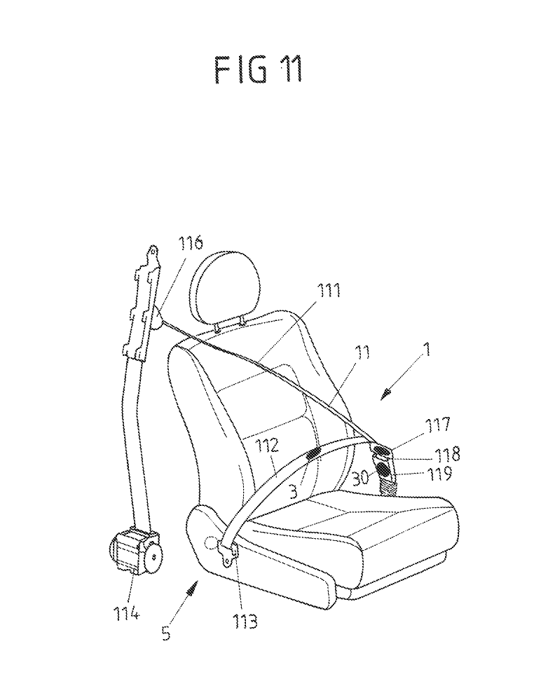

FIG. 10 illustrates an exemplary embodiment of the method according to the invention for determining information with respect to the cardiac and/or respiratory activity of the vehicle occupant. Accordingly, there is used a safety belt arrangement according to the invention which comprises at least three sensor units 3a, 3b, 3c. The sensor unit 3a includes at least one acceleration sensor as well as an audio sensor, the sensor unit 3b includes at least one audio sensor, and the sensor unit 3c includes at least one audio sensor as well as an (in particular capacitive) ECG sensor.

Correspondingly, the sensor unit 3a generates a sensor signal 301 dependent on the movement (acceleration) of the vehicle occupant (in particular of the chest of the vehicle occupant) as well as a sensor signal dependent on the heart and/or breathing sound of the vehicle occupant. Analogously, the sensor units 3b, 3c each generate a sensor signal dependent on a heart and/or breathing sound, which is put together with the signal of the audio sensor of the sensor unit 3a to obtain a combined (in particular processed) audio signal 302. The composite audio signal 302 is split up into two signals 302a, 302b which are associated to the breathing sound or the heart sound. By means of the ECG sensor of the sensor unit 3a an ECG signal 303 is generated.

The movement sensor signal 301 is subjected to a movement analysis 304, in order to determine information 305 on the respiration of the vehicle occupant (e.g. a respiratory frequency). Furthermore, a heart rate 306 is determined by means of the movement analysis 304. By taking account of the respiratory sinus arrythmia—RSA 307—of the heart rate, information 308 on the respiration (e.g. likewise a respiratory frequency) in turn can be determined from the heart rate 306.

The audio signal 302a is subjected to a breathing sound analysis, in order to likewise obtain information 309 on the respiration of the vehicle occupant. The audio signal 302b is evaluated by a heart sound analysis, in order to determine a heart rate 310. Analogous to steps 307 and 308 an evaluation 311 of the respiratory sinus arrythmia of the heart rate 310 is made, which provides information 312 on the respiration of the vehicle occupant.

By means of an evaluation of the ECG signal 303 there is likewise obtained a heart rate 313, from which in turn information 315 on the respiration can be derived by means of an evaluation 314 of the respiratory sinus arrythmia.

The sensors of the sensor units 3a, 3b, 3c thus provide information 305, 308, 309, 312, 315 on the respiration in five different ways. This information is subjected to a plausibility check and e.g. also to a signal processing and there is generated a plausibilized and/or more exact respiratory signal 316 (in particular a respiratory frequency). For example, the plausibility check comprises a comparison and a selection of some of the information 305, 308, 309, 312, 315. The sensors also supply various measurement values of the heart rate, namely the heart rates 306, 310. These measurement values likewise are combined and/or plausibilized, in order to generate a plausibilized and/or more exact heart rate 317. The plausibilized and/or more exact respiratory frequency information 316 and/or heart rate 317 are utilized for the further processing, in particular to derive information with respect to the mental and/or physical state of the vehicle occupant.

The method explained with reference to FIG. 10 can be carried out in particular by using the safety belt arrangement of FIGS. 1 to 6.

FIG. 11 shows another exemplary embodiment of the safety belt arrangement 1 according to the invention in a perspective view. Accordingly, the safety belt arrangement 1 is formed in the form of a three-point safety belt system and associated to a vehicle seat 5. The safety belt arrangement 1 comprises a safety belt 11 which is at least partly retracted in a vehicle-fixed belt retractor 114 or is stored there, wherein the safety belt 11 can be extracted from the belt retractor 114.

When the safety belt 11 is fastened at a vehicle occupant (not shown) occupying the vehicle seat 5, it extends proceeding from the belt retractor 114 along the vertical vehicle axis upwards to a vehicle-fixed shoulder deflection fitting 116 which can be formed vertically adjustable along the vertical vehicle axis. The safety belt 11 is deflected by means of this shoulder deflection fitting 116 and then extends along the vertical vehicle axis diagonally across the upper body of the vehicle occupant sitting on the vehicle seat 5 downwards to a buckle deflection element 117.

At the buckle deflection element 117 a belt buckle tongue 118 is formed, which in the condition of the vehicle occupant buckled up is inserted into a belt buckle 119 fixed at the vehicle and thereby is releasably arrested in the same. The safety belt 11 is deflected by the buckle deflection element 117 and extends across a lap region of the vehicle occupant to a vehicle-fixed belt anchor 113.

The belt deflection means (the buckle deflection element 117), which along the vertical vehicle axis is arranged approximately at the height of the hip of a vehicle occupant occupying the seat, divides the safety belt 11 into two belt segments, namely into a shoulder segment 111 (upper body portion or shoulder belt), which proceeding from the shoulder deflection fitting 116 extends diagonally across the upper body of the vehicle occupant buckled up to the buckle deflection element 117. The shoulder segment 111 is followed by a lap segment 112 (lap belt), which proceeding from the buckle deflection element 117 extends to the belt anchorage 113.

At the lap segment 112 of the safety belt 11 a sensor region in the form of a sensor unit 3 is arranged. As already explained above in particular in conjunction with the other exemplary embodiments, the sensor unit includes at least two sensors which serve to determine data with respect to the cardiac and/or respiratory activity of the vehicle occupant.

In addition, a further sensor unit 30 is provided at the belt buckle 119. The sensor unit 30 likewise is equipped with at least two sensors, as explained above (cf. in particular FIGS. 3A and 3B). It is also conceivable that the sensor unit 30 is not located at the belt buckle 119, but at the belt tongue 118. It is also possible that both at the belt buckle 119 and at the belt tongue 118 at least one sensor unit 30 each is arranged. Of course, several sensor units each can be provided; in particular, the safety belt 11 can include several sensor units (also in the region of the shoulder segment 111). In one modification, only the safety belt 11 includes a sensor unit (e.g. exactly one). On the other hand, a sensor unit can of course also be present only at the belt buckle 119 and/or at the belt tongue 118 (i.e. the safety belt 11 has no sensor unit).

### LIST OF REFERENCE NUMERALS

1 safety belt arrangement
2 vehicle occupant
3, 3a, 3b, 3c, 30 sensor unit
4 evaluation unit
5 vehicle seat
6 printed circuit board
11 safety belt
11a inside
11b outside
31, 32, 33 sensor
34 amplification and/or filter unit
35 connecting line
36 electric line
51 backrest
111 upper body portion
112 lap portion
113 belt anchor
114 retractor
115 tubular receptacle
116 shoulder deflection fitting
117 buckle deflection element
118 belt tongue
119 belt buckle

1. A safety belt arrangement for a motor vehicle, comprising a safety belt, a belt buckle, and a belt tongue; at least one sensor region arranged at the safety belt, at the belt buckle and/or at the belt tongue, which includes: at least one first sensor for determining values of a quantity dependent on a cardiac and/or respiratory activity of a user of the safety belt; and at least one second sensor for determining values of at least one ambient quantity,
and at least one evaluation unit for determining information with respect to the cardiac and/or respiratory activity of the user of the safety belt by using the values determined by the first and second sensors of the sensor region.

2. The safety belt arrangement according to claim 1, further comprising a plurality of sensor regions arranged at the safety belt, at the belt buckle and/or at the belt tongue, wherein the sensor regions each include at least one first sensor for determining values of a quantity dependent on a cardiac and/or respiratory activity of a user of the safety belt and at least one second sensor for determining values of at least one ambient quantity, and wherein the evaluation unit is formed for determining information with respect to the cardiac and/or respiratory activity of the user of the safety belt by using the values determined by the first and second sensors of at least one of the sensor regions.

3. The safety belt arrangement according to claim 2, wherein several sensor regions are provided at the safety belt.

4. The safety belt arrangement according to claim 2, wherein at least one sensor region is provided at the safety belt and a further sensor region is provided at the belt buckle or the belt tongue.

5. The safety belt arrangement according to claim 1, wherein the first sensor is formed by a first acoustic transducer and the second sensor is formed by a second acoustic transducer, wherein the first and the second acoustic transducer are oriented along different directions.

6. The safety belt arrangement according to claim 1, wherein in use of the safety belt arrangement the first acoustic transducer is directed towards the user of the safety belt arrangement, whereas the second acoustic transducer is oriented such that it detects sounds of the surroundings of the sensor region.

7. The safety belt arrangement according to claim 5, wherein the evaluation unit is formed to correct signals which are generated by the first acoustic transducer on detection of sounds which depend on the cardiac and/or respiratory activity of the user, by using surrounding sounds detected with the second acoustic transducer.

8. The safety belt arrangement according to claim 7, wherein the correction is made by using a model of an acoustic transmission path between the first and the second acoustic transducer.

9. The safety belt arrangement according to claim 1, wherein the first sensor is an acceleration sensor or a pressure sensor.

10. The safety belt arrangement according to claim 9, wherein the second sensor is a temperature sensor which detects a temperature in the surroundings of the first sensor.

11. The safety belt arrangement according to claim 10, wherein the evaluation unit is formed to correct signals which are generated by the first sensor in dependence on the cardiac and/or respiratory activity of the user, by using the temperature detected with the temperature sensor.

12. A safety belt arrangement for a motor vehicle, comprising a safety belt, a belt buckle, and a belt tongue; at least one sensor region arranged at the safety belt, at the belt buckle and/or at the belt tongue, which includes: at least one first sensor for determining values of a first quantity dependent on a cardiac and/or respiratory activity of a user of the safety belt; and at least one second sensor for determining values of a second quantity dependent on a cardiac and/or respiratory activity of the user of the safety belt and different from the first quantity,
and at least one evaluation unit for determining information with respect to the cardiac and/or respiratory activity of the user of the safety belt by using the values determined by the first and second sensors of the sensor region.

13. The safety belt arrangement according to claim 12, further comprising a plurality of sensor regions arranged at the safety belt, at the belt buckle and/or at the belt tongue, wherein the sensor regions each include at least one first sensor for determining values of a first quantity dependent on a cardiac and/or respiratory activity of a user of the safety belt and at least one second sensor for determining values of at least one second quantity dependent on a cardiac and/or respiratory activity of the user of the safety belt and different from the first quantity, and wherein the evaluation unit is formed for determining information with respect to the cardiac and/or respiratory activity of the user of the safety belt by using the values determined by the first and second sensors of at least one of the sensor regions.

14. The safety belt arrangement according to claim 13, wherein several sensor regions are provided at the safety belt.

15. The safety belt arrangement according to claim 13, wherein at least one sensor region is provided at the safety belt and a further sensor region is provided at the belt buckle or the belt tongue.

16. The safety belt arrangement according to claim 15, wherein the evaluation unit is formed to correct a signal of the first sensor in dependence on a signal of the second sensor.

17. The safety belt arrangement according to claim 1, wherein the first and/or the second sensor each are arranged on a printed circuit board extending along at least a portion of the safety belt.

18. The safety belt arrangement according to claim 17, wherein that the printed circuit board is arranged in a tubular receptacle of the safety belt.

19. The safety belt arrangement according to claim 1, wherein the evaluation unit is formed to generate a signal in dependence on the values each determined by the first and/or second sensor in the sensor regions, which signalizes whether the safety belt is fastened correctly.

20. A method for determining information with respect to the cardiac and/or respiratory activity of a user of a safety belt arrangement of a motor vehicle, with the following steps: providing a safety belt, a belt buckle, a belt tongue and at least one sensor region arranged at the safety belt, the belt buckle and/or the belt tongue, which includes: at least one first sensor for determining values of a first quantity dependent on a cardiac and/or respiratory activity of a user of the safety belt; and at least one second sensor for determining values of at least one ambient quantity,
determining information with respect to the cardiac and/or respiratory activity of the user of the safety belt by using the values determined by the first and second sensors of the sensor region.

21. A method for determining information with respect to the cardiac and/or respiratory activity of a user of a safety belt arrangement of a motor vehicle, with the following steps: providing a safety belt, a belt buckle, a belt tongue and at least one sensor region arranged at the safety belt, the belt buckle and/or the belt tongue, which includes: at least one first sensor for determining values of a first quantity dependent on a cardiac and/or respiratory activity of a user of the safety belt; and at least one second sensor for determining values of a second quantity dependent on a cardiac and/or respiratory activity of the user of the safety belt and different from the first quantity,
determining information with respect to the cardiac and/or respiratory activity of the user of the safety belt by using the values determined by the first and second sensors of the sensor region.
